(19) United States
(12) Patent Application Publication
(10) Pub. No.: US 2025/0188027 A1
(43) Pub. Date: Jun. 12, 2025

(54) PROCESS FOR THE PRODUCTION OF A TERTIARY AMINE SURFACTANT

(71) Applicant: BASF SE, Ludwigshafen Am Rhein (DE)

(72) Inventors: Sylvester GROESSL, Ludwigshafen (DE); Juergen TROPSCH, Ludwigshafen (DE); Marcel Patrik KIENLE, Limburgerhof (DE); Sandra HOMANN, Ludwigshafen (DE); Dagmar Pascale KUNSMANN-KEITEL, Ludwigshafen (DE)

(21) Appl. No.: 18/846,324

(22) PCT Filed: Mar. 30, 2023
(86) PCT No.: PCT/EP23/58285

§ 371 Date: Sep. 12, 2024

(30) Foreign Application Priority Data
Mar. 30, 2022 (EP) 22165296.9

(51) Int. Cl. C07D 207/04; B01J 21/18; B01J 23/44; C11D 1/42; C11D 1/62

(52) U.S. Cl. CPC C07D 207/04; B01J 21/18; B01J 23/44; C11D 1/42; C11D 1/62

(57) ABSTRACT

Described herein is a process for the production of a surfactant, as well as a surfactant composition. Also described herein are specific surfactants and compositions thereof, as well as methods of their use in a wide variety of applications such as all purpose cleaning agents.

The present invention relates to a process for the production of a tertiary amine surfactant. The invention further relates to individual embodiments of the surfactant, as well as surfactant compositions.

Surfactants are compounds which lower the surface tension between two phases, in particular between two liquids. Surfactants generally are organic amphiphilic compounds, i.e., compounds comprising both hydrophobic and hydrophilic groups. Such compounds find use, e.g., as detergents, wetting agents, foaming agents, emulsifiers, and dispersants.

N-alkyl amino-based surfactants have shown promise as amphoteric surfactants. These surfactants have amine moieties as polar headgroups. N-alkyl amino-based surfactants exhibit low critical micelle concentrations (CMCs) and provide low values of surface tension at the CMC.

Being commodities, surfactants are produced on a large scale, and there is a pressing need to develop direct sustainable catalytic methods to obtain environmentally friendly and fully bio-based alternatives, especially in the context of a bio-based economy. Despite the obvious potential of N-alkyl amino compounds, there is a relative lack of selective catalytic methods to obtain these compounds via direct functionalization of unprotected amino compounds such as secondary amines.

In general, N-alkylation of secondary amines has been performed using stoichiometric methods, such as reductive amination of aldehydes with complexing salts, or nucleophilic substitution with alkyl halides. One method described in the art comprises reductive amination of fatty aldehydes with secondary amines in the presence of elemental hydrogen.

JP-S-62149647 A describes the reductive amination of n-dodecanal with morpholine at a temperature of 150 to 250° C. and a molecular hydrogen pressure of 5 bar in the presence of a heterogeneous catalyst selected from palladium, platinum, Raney-copper and Raney-Nickel.

The selective N-monoalkylation of secondary amines under mild conditions remains challenging. For instance, the use of alkyl halides can lead to exhaustive alkylation (peralkylation) of the nitrogen center resulting in quaternary ammonia salts.

There remains a need for providing surfactants under environmentally beneficial conditions at high yield, in particular using bio-renewable starting materials, i.e., materials which are naturally available, which are available through biological processes, and/or which are available through chemical processes from naturally available materials. There is moreover a need for novel surfactants.

The present invention provides a process for the production of a tertiary amine surfactant of general formula (I)

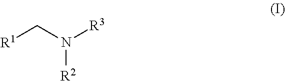

or a salt thereof, wherein
R¹ is selected from C₅-C₁₇-alkyl and C₅-C₁₇-alkenyl;
R² and R³ are independently selected from C₁-C₆-alkyl and C₃-C₈-alkenyl, which C₁-C₈-alkyl and C₃-C₈-alkenyl is each optionally substituted with one or more substituents selected from a hydroxy group, a C₁-C₈-alkoxy group, an amino group, a C₁-C₈-alkylamino group, a thio group and a C₁-C₈-alkylthio group;
or wherein R² and R³, together with the nitrogen atom to which they are bound, form a 5- or 6-membered heterocycle, optionally substituted with one or more substituents selected from a hydroxy group, a C₁-C₈-alkoxy group, an amino group, a C₁-C₈-alkylamino group, a thio group and a C₁-C₈-alkylthio group;
the process comprising reductive amination of an aldehyde of general formula (II)

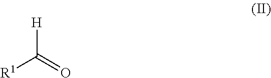

wherein R¹ is defined as above;
with a secondary amine of general formula (III)

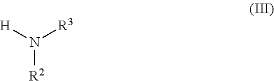

wherein R² and R³ are defined as above;
in the presence of molecular hydrogen and a heterogeneous catalyst comprising a group 10 element of the periodic table of the elements;
wherein the reductive amination is conducted in feed operation, wherein the secondary amine of general formula (III) is provided, and the aldehyde of general formula (II) is metered thereto; and
wherein the reductive amination is performed
   at a pressure of molecular hydrogen in the range of at least 1 bara to less than 40 bara, preferably in the range of more than 1 bara to less than 30 bara and
   at a temperature in the range of 25 to 70° C.

The process allows for high yields under mild reaction conditions. Moreover, the aldehydes of formula (II) and the secondary amines of formula (III) are typically naturally available, available through biological processes, and/or available through chemical processes from naturally available materials. Overall, the process thus allows for obtaining surfactants in an environmentally friendly fashion.

The process for the production of the tertiary amine surfactant of general formula (I) comprises reductive amination of an aldehyde of general formula (II) with a secondary amine of general formula (III):

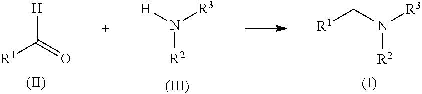

R¹ is selected from C₅-C₁₇-alkyl and C₅-C₁₇-alkenyl, and may be straight-chained or branched. When R¹ is an alkenyl group, R¹ may be monounsaturated or polyunsaturated. R¹ is preferably selected from C₇-C₁₅-alkyl and C₇-C₁₅-alkenyl, more preferably from C₇-C₁₃-alkyl and C₇-C₁₃-alkenyl, most preferably from C₇-C₁₁-alkyl and C₇-C₁₁-alkenyl.

In a particularly preferred embodiment, R¹ is a C₈-, C₉- or C₁₁-alkyl, especially a C₈- or C₁₁-alkyl; or R¹ is a C₉-C₁₇-alkenyl, in particular C₉-alkenyl.

The aldehyde of general formula (II) is an aliphatic aldehyde. Suitable aldehydes of general formula (II) include octanal, nonanal, decanal, undecanal, dodecanal, tetradecanal, hexadecanal, 2-ethylhexanal and citral, in particular citral and dodecanal. Citral is understood as a mixture of two geometric isomers, specifically geranial (citral A) and neral (citral B).

R² and R³ may be independently selected from C₁-C₈-alkyl and C₃-C₈-alkenyl, which may each be straight-chained or branched. Preferably, R² and R³ are independently selected from optionally substituted C₁-C₈-alkyl, especially C₁-C₆-alkyl and optionally substituted C₁-C₄-alkyl, in particular from optionally substituted methyl, optionally substituted ethyl, optionally substituted n-propyl and optionally substituted n-butyl. In a preferred embodiment, R² is optionally substituted C₁-C₃-alkyl, in particular C₁-C₃-alkyl substituted with a hydroxy group, and R³ is optionally substituted C₁-C₃-alkyl, in particular methyl. In a particularly preferred embodiment, R² is hydroxyethyl and R³ is methyl.

R² and R³ are each independently optionally substituted with one or more substituents, such as 1 to 3 substituents, selected from a hydroxy group, a C₁-C₈-alkoxy group, an amino group, a C₁-C₈-alkylamino group, a thio group and a C₁-C₈-alkylthio group, in particular a hydroxy group.

In a preferred embodiment, R² and R³, together with the nitrogen atom to which R² and R³ are bound, form a 5- or 6-membered heterocycle, such as a pyrrolidinyl group, a piperidinyl group, a morpholinyl group, or a 8-oxa-3-azabicyclo[3.2.1]octanyl group. The heterocycle may be saturated or unsaturated, preferably saturated. The heterocycle is optionally substituted with a hydroxy group, a C₁-C₈-alkoxy group, an amino group, a C₁-C₈-alkylamino group, a thio group or a C₁-C₈-alkylthio group.

Suitable secondary amines of general formula (III) include pyrrolidine, piperidine, morpholine, 8-oxa-3-azabicyclo[3.2.1]octane and N-methyl ethanolamine.

The molar ratio of the total amount of the aldehyde of general formula (II) to the total amount of the secondary amine of general formula (III) is preferably in the range of 0.8:1 to 1.3:1, more preferably 0.9:1 to 1.2:1, most preferably 0.95:1 to 1.15:1.

The reductive amination is performed in the presence of a heterogeneous catalyst comprising a group 10 element of the periodic table of the elements. The group 10 element may be selected from nickel (Ni), palladium (Pd) and platinum (Pt). Preferably, the group 10 element is selected from nickel (Ni), platinum (Pt) and palladium (Pd), more preferably from platinum (Pt) and palladium (Pd), in particular palladium (Pd).

The metals can be used as such, or else applied to supports. Preferred supports are selected from aluminum oxide, silicon dioxide, titanium dioxide, zirconium dioxide and activated carbon. Particular preference is given to the supported metals. Preferred supports are activated carbon, aluminum oxide and titanium dioxide. A very particularly preferred support is activated carbon. A very especially preferred hydrogenation catalyst is palladium on activated carbon.

In a preferred embodiment, the heterogeneous catalyst is selected from Raney nickel and palladium on activated carbon, in particular palladium on activated carbon (Pd/C). When the heterogeneous catalyst is palladium on activated carbon, the catalyst may comprise a binder such as polytetrafluoroethylene (PTFE).

The reductive amination is performed at a pressure of molecular hydrogen in the range of at least 1 bara to less than 40 bara, preferably in the range of at least 1 bara to less than 35 bara, in particular more than 1 bara to 30 bara. Preferably, the pressure of molecular hydrogen is in the range of 3 to 30 bara, more preferably 3 to 25 bara, most preferably 4 to 21 bara. A pressure of molecular hydrogen of more than 1 bara allows for a favorably low reaction time, thus increasing the efficiency of the process.

On the other hand, if the pressure of molecular hydrogen is too high, the aldehyde is reduced to the corresponding alcohol, rather than partaking in the reductive amination. The corresponding alcohol is notably difficult to separate from the reaction mixture. Thus, working at a pressure of molecular hydrogen within the claimed range allows for high selectivity and high efficiency of the process.

The reductive amination is performed at a temperature of 25 to 70° C. Preferably, the temperature is in the range of 25 to 65° C., more preferably 30 to 60° C., most preferably 40 to 50° C. A temperature in this range allows for the reductive amination reaction to proceed at an adequate rate under mild conditions.

The reductive amination is typically performed in the presence of a solvent. A solvent may bring advantages to the reductive amination reaction, including an improved hydrogen solubility, a decreased viscosity of the reaction mixture, an improved mixing efficiency, and an improved heat transfer.

In one embodiment, the reductive amination is performed in the presence of a solvent having a Hildebrand solubility parameter δ in the range of 18 to 38 MPa^{1/2}, preferably in the range of 25 to 37 MPa^{1/2}, more preferably 26 to 36 MPa^{1/2}, most preferably 30 to 36 MPa^{1/2}, in particular 32 to 35 MPa^{1/2}. It was found that a solvent having a Hildebrand solubility parameter δ in this range allows for a high solubility of both the starting materials and the obtained surfactant. The solvent may be in the form of a single solvent or a mixture of two or more solvents. The Hildebrand solubility parameter δ provides a numerical estimate of the degree of interaction between compounds, and thus be used as an indicator of solubility. Compounds having similar values of δ are likely miscible.

The Hildebrand solubility parameter δ is well-known in literature. The following table provides exemplary solvents and their Hildebrand solubility parameter from Barton, Allan F. M. (1983), Handbook of Solubility Parameters and Other Cohesion Parameters, CRC Press.

| solvent | δ [MPa^{1/2}] |
|---|---|
| n-pentane | 14.4 |
| n-hexane | 14.9 |
| n-Heptane | 15.3 |
| diethyl ether | 15.4 |
| n-dodecane | 16.0 |
| cyclohexane | 16.8 |
| xylene | 18.2 |
| ethyl acetate | 18.2 |
| toluene | 18.3 |
| tetrahydrofuran | 18.5 |
| benzene | 18.7 |
| chloroform | 18.7 |
| methyl ethyl ketone | 19.3 |
| acetone | 19.7 |
| ethylene dichloride | 20.2 |
| methylene chloride | 20.2 |
| pyridine | 21.7 |
| morpholine | 22.1 |
| dimethylformamide | 24.7 |
| n-propanol | 24.9 |
| ethanol | 26.2 |
| dimethyl sulfoxide | 26.4 |
| n-butanol | 28.7 |
| methanol | 29.7 |
| propylene glycol | 30.7 |
| ethylene glycol | 34.9 |
| glycerol | 36.2 |
| water | 48.0 |

The Hildebrand solubility parameter δ of a solvent mixture may be determined by averaging the Hildebrand values of the individual solvents by volume. For example, the Hildebrand solubility parameter of a mixture of two parts ethanol and one part water may be calculated as:

$$δ = ( ( 2 \times 26.2 {MPa}^{1 / 2} + 48. {MPa}^{1 / 2} ) / 3 ) = 33.5 {MPa}^{1 / 2} .$$

Selection of a solvent having a Hildebrand solubility parameter δ in the above range ensures that the solvent has solubility characteristics suitable for the secondary amine of general formula (III), and that the reductive amination reaction proceeds at an adequate rate.

In a preferred embodiment, the solvent comprises a protic solvent and/or an aprotic solvent comprising an ether moiety, in particular a protic solvent. A protic solvent is understood to be a solvent which is able to donate a proton (H⁺) to a solute, e.g., via an O—H or N—H bond, typically via hydrogen bonding. An aprotic solvent is unable to donate a proton to a solute.

Preferably, the solvent comprises at least one protic solvent selected from water and an aliphatic alcohol. Preferred aliphatic alcohols include methanol, ethanol and isopropanol, in particular ethanol. More preferably, the solvent comprises an aliphatic alcohol or a mixture of an aliphatic alcohol and water. In another preferred embodiment, the solvent is a mixture of an aliphatic alcohol and water in a volume ratio in the range of 1:1 to 4:1, preferably 1.25:1 to 3:1, most preferably 1.5:1 to 2:1. In a particularly preferred embodiment, the solvent is a mixture of ethanol and water in a volume ratio in the range of 1:1 to 4:1, preferably 1.25:1 to 4:1, more preferably 1.5:1 to 2:1, most preferably 1.5:1 to 2:1. When the solvent is composed of a mixture of different solvents, the volume of the mixture may be smaller than the sum of the volumes of the individual solvents. This phenomenon is known as “volume contraction”. It may occur, e.g., when the solvent is composed of an aliphatic alcohol and water. The volume ratio is understood to relate to the ratio of the individual solvent volumes prior to mixing, i.e., disregarding any volume contraction which may occur upon mixing.

In another embodiment, the solvent comprises at least one aprotic solvent comprising an ether moiety, such as tetrahydrofuran, 2-methyltetrahydrofuran, 1,4-dioxane, or tetrahydropyran, in particular tetrahydrofuran. In one embodiment, the solvent is tetrahydrofuran.

In a further embodiment, the solvent comprises at least one aprotic solvent comprising an ether moiety and at least one protic solvent. For example, the solvent may comprise a mixture of tetrahydrofuran with at least one protic solvent, in particular water or an alcohol. In a preferred embodiment, the solvent is a mixture of tetrahydrofuran with water or ethanol, in particular a mixture of tetrahydrofuran with water.

Additionally, the preferred solvents may prevent the occurrence of two separate liquid phases during the reductive amination, which is possible due to liberation of water as the co-product in the reaction.

The reductive amination is preferably performed at a solvent dilution ratio of less than 5.0 L per kg. The term solvent dilution ratio as used herein refers to the total volume of solvent to the total weight of the aldehyde of general formula (II) and the secondary amine of general formula (III). The term “total volume of solvent” includes the initial volume of solvent as well as solvent volumes added over the course of the reductive amination. A solvent volume added over the course of the reductive amination is, for example, the solvent volume contained in a solution of a reactant that is metered in during a feed operation process. It is understood that the “total volume of solvent” takes into account the volume contraction.

Preferably, the reductive amination is performed at a solvent dilution ratio of at most 5.0 L per kg, more at most 3.5 L per kg. Typically, the reductive amination is performed at a solvent dilution ratio of 1.0 to 4.5 L per kg, more preferably 1.0 to 3.5 L per kg. A concentration of the aldehyde of general formula (II) and the secondary amine of general formula (III) in this range allows for efficiently reacting the secondary amine with the aldehyde at high selectivity. More diluted reaction mixtures result in poor space-time yields, while in some cases of too highly concentrated reaction mixtures, the benefits of the solvent may be minimized.

The process of the invention may be carried out in any reactor suitable for maintaining the pressure of molecular hydrogen in the desired range and at the desired temperature, such as an autoclave.

The process of the invention is conducted in feed operation, wherein the secondary amine of general formula (III) is provided (i.e., initially charged), and the aldehyde of general formula (II) is metered thereto. The reductive amination may occur in the presence of a solvent. It is understood that providing the secondary amine of general formula (III) also includes providing a solution of the secondary amine of general formula (III). Likewise, metering the aldehyde of general formula (II) thereto includes metering a solution of the aldehyde of general formula (II) thereto. The solvent contained in the solution of the secondary amine of general formula (III) and the solvent contained in the solution of the aldehyde of general formula (II) together constitute the solvent in the presence of which the reductive amination occurs. The solvents comprised in the two solutions may be identical or different. In the latter case, the solvent in the presence of which the reductive amination occurs will be a mixture of solvents.

In general, the aldehyde of general formula (II) is added at constant feed rate. This allows for avoiding undesired side reactions. Thus, on the one hand, the reduction of the aldehyde to the corresponding alcohol is suppressed, obtaining the tertiary amine surfactant of general formula (I) at higher selectivity and avoiding the removal of the difficult to separate corresponding alcohol. On the other hand, aldol condensation of the aldehyde with itself to form the aldehyde dimer is avoided. This again allows for higher selectivity and moreover suppresses the formation of dimer-based surfactants with less advantageous properties.

The reaction product obtained in the process of the invention may be purified by typical means, such as filtration of the reaction mixture to remove the heterogeneous catalyst and subsequent evaporation of the solvent from the filtrate to obtain a crude material. In order to fully solubilize the reaction product in the obtained reaction mixture prior to filtration, a suitable additional solvent, in particular ethanol, may be added. The crude material may be further purified by, e.g., distillation.

The surfactant may be a non-ionic compound of general formula (I) or an external salt of a compound of general formula (I). “External salts” may be obtained under acidic conditions. In one embodiment, the process comprises neutralizing the tertiary amine surfactant of general formula (I) with an acid. Thus, a salt of the tertiary amine surfactant of general formula (I) is obtained. The acid may be selected from organic acids and mineral acids.

Suitable mineral acids include hydrochloric acid, sulfuric acid, amidosulfuric acid, and phosphoric acid. Preferably, the mineral acid is selected from hydrochloric acid and sulfuric acid.

Suitable organic acids include carboxylic acids, sulfonic acids, carbonic acid, organic phosphonic acids, and aminocarboxylic acids. Preferably, the organic acid is selected from carboxylic acids, most preferably from formic acid, acetic acid, oxalic acid, propionic acid, hydroxypropionic acid, lactic acid, malic acid, maleic acid, succinic acid, tartaric acid, citric acid, aconitic acid and glutamic acid.

Neutralization of the tertiary amine surfactant of general formula (I) with an acid is typically performed at room temperature, i.e., 25° C., and under atmospheric pressure. Neutralization is typically carried out in a solvent, preferably a solvent as described above. The neutralized reaction product may be purified by typical means, such as evaporation of the solvent to obtain a crude material, which may be further purified by, e.g., recrystallization.

In one embodiment, the aldehyde of general formula (II) and/or the secondary amine of general formula (III) are bio-based compounds, in particular the aldehyde of general formula (II). In this embodiment, the process allows for obtaining surfactants in a particularly environmentally friendly fashion.

Using radiocarbon and isotope ratio mass spectrometry analysis, the bio-based content of materials can be determined. For example, ASTM International has established a standard method for assessing the bio-based content of materials. The ASTM method is designated ASTM-D6866.

The application of ASTM-D6866 to determine the “bio-based content” of materials is built on the same concepts as radiocarbon dating, but without use of the age equations. The analysis is performed by deriving a ratio of the amount of radiocarbon (¹⁴C) in an unknown sample to that of a modem reference standard. The ratio is reported as a percentage with the units “pMC” (percent modern carbon). If the material being analyzed is a mixture of present day radiocarbon and fossil carbon (containing no radiocarbon), then the pMC value obtained correlates directly to the amount of Biomass material present in the sample.

The modern reference standard used in radiocarbon dating is a NIST standard with a known radiocarbon content equivalent approximately to the year AD 1950. AD 1950 was chosen since it represented a time prior to thermo-nuclear weapons testing which introduced large amounts of excess radiocarbon into the atmosphere with each explosion (termed “bomb carbon”). The AD 1950 reference represents 100 pMC.

Further details for the assessment of materials with regard to determining whether or not they are bio-based may be found, e.g., in WO 2007/095262 A2.

The surfactants of general formula (I) are preferably at least partially biodegradable. Biodegradation is preferably at least 20%, more preferably at least 60% (all percentages in wt.-% based on the total solid content) within 28 days according to OECD 301.

It is believed that while the present process allows for avoiding a detrimentally high degree of aldol condensation of the aldehyde with itself to form the aldehyde dimer, and thus allows for higher selectivity, dimerization cannot be avoided altogether. It is believed that a small amount of dimer-based surfactants may have an advantageous effect on important surfactant properties, such as foam volume, wetting time, surface tension and contact angle.

The present invention thus further provides a surfactant composition, comprising a surfactant of general formula (I) or a salt thereof as defined above in an amount of at least 90 wt.-%, relative to the weight of the surfactant composition, and a compound of general formula (IV)

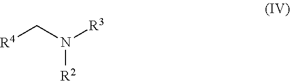

or a salt thereof in an amount of 0.01 to 5.0 wt.-%, relative to the weight of the surfactant composition,
wherein R² and R³ are defined as above; and R⁴ is selected from C₁₀-C₃₄-alkyl and C₁₀-C₃₄-alkenyl, with the proviso that the number of carbon atoms in R⁴ differs from the number of carbon atoms in R¹ of the surfactant of general formula (I), wherein the number of carbon atoms in R⁴ is preferably greater than the number of carbon atoms in R¹ in the surfactant of general formula (I), for example twice the number of carbon atoms in R¹ in the surfactant of general formula (I);
wherein the compound of general formula (IV) is preferably obtained via the reductive amination of an aldehyde dimer obtained from the self-aldol condensation of an aldehyde of formula (II) as defined above and a secondary amine of general formula (III) as defined above.

In one embodiment, the surfactant composition comprises the compound of general formula (IV) in an amount of 0.01 to 4.5 wt.-%, preferably 0.05 to 4.5 wt.-%, more preferably 0.1 to 4.0 wt.-%, most preferably 0.5 to 3.0 wt.-%, relative to the weight of the surfactant composition.

In one embodiment, the surfactant composition comprises the surfactant of general formula (I) in an amount of at least 93 wt.-%, preferably at least 94 wt.-%, more preferably at least 95 wt.-%, such as at least 96 wt.-% or at least 97 wt.-%, relative to the weight of the surfactant composition.

The surfactant composition may further comprise side products of the process of the invention, such as unreacted starting materials or side products, e.g., alcohols.

Preferably, the surfactant composition comprises less than 5 wt.-%, more preferably less than 4 wt.-%, most preferably less than 3 wt.-%, of compounds besides the surfactant of general formula (I) and the compound of general formula (IV).

Preferably, the surfactant composition is obtained according to a process of the invention. The surfactant composition may be admixed with further components so as to obtain surfactants for a wide variety of applications, including detergents, as described in detail below.

In another embodiment, the present invention further provides a surfactant composition, comprising a surfactant of general formula (I) or a salt thereof as defined above and a compound of general formula (IV)

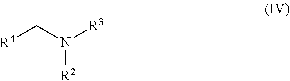

or a salt thereof,
wherein R² and R³ are defined as above; and R⁴ is selected from C₁₀-C₃₄-alkyl and C₁₀-C₃₄-alkenyl, with the proviso that the number of carbon atoms in R⁴ differs from the number of carbon atoms in R¹ of the surfactant of general formula (I), wherein the number of carbon atoms in R⁴ is preferably greater than the number of carbon atoms in R¹ in the surfactant of general formula (I), for example twice the number of carbon atoms in R¹ in the surfactant of general formula (I);
wherein the weight ratio of the total amount of surfactant of general formula (I) and salts thereof to the total amount of compound of general formula (IV) and salts thereof is in the range of 15:1 to 10,000:1, preferably 20:1 to 1,000:1, more preferably 25:1 to 100:1, such as 25:1 to 75:1;
wherein the compound of general formula (IV) is preferably obtained via the reductive amination of an aldehyde dimer obtained from the self-aldol condensation of an aldehyde of formula (II) as defined above and a secondary amine of general formula (III) as defined above.

In the process of the invention, the surfactant of general formula (I) is obtained, together with a compound of general formula (IV) as a side product in small amounts. Preferably, the compound of general formula (IV) comprised in the surfactant compositions of the invention is derived from the self-aldol condensation product of the aldehyde (II) used in the process of the invention. The compound of general formula (IV) may be obtained in an enriched form from the crude reaction product of the process of the invention via, e.g., distillation. The proportions of the surfactant of general formula (I) and the compound of general formula (IV) may be determined via gas chromatography (after silylation) or via gas chromatography-mass spectrometry (GC-MS).

The invention moreover provides a compound selected from
2-[(3,7-dimethyloctyl)(methyl)amino]ethanol,
1-(3,7-dimethyloctyl)pyrrolidine,
3-dodecyl-8-oxa-3-azabicyclo[3.2.1]octane,
3-nonyl-8-oxa-3-azabicyclo[3.2.1]octane, and
3-(3,7-dimethyloctyl)-8-oxa-3-azabicyclo[3.2.1]octane,
or a salt thereof.

These compounds may be obtained by the process of the invention.

The invention moreover provides a composition comprising at least one compound of the invention.

The invention further provides the use of a compound of the invention or of a composition of the invention as a surfactant. The surfactants and compositions of the invention, which are understood to include the surfactant compositions of the invention, can be used as surfactants in a wide variety of applications, including detergents such as granular laundry detergents, liquid laundry detergents, liquid dishwashing detergents; and in miscellaneous formulations such as all purpose cleaning agents, liquid soaps, shampoos, shower gels, and liquid scouring agents. Moreover, the compounds and compositions of the invention may be used as surfactants in institutional and industrial cleaning formulations such as kitchen cleaners, industrial laundry detergents, vehicle cleaners, and disinfection cleaners. Moreover, the compounds and compositions of the invention may be used as adjuvants in agrochemical formulations, such as pesticide formulations.

The compounds and compositions of the invention find particular use as surfactants in detergents, specifically laundry detergents and manual dishwashing products. These are generally comprised of a number of components besides the compound(s) of the invention. The composition typically comprises a total amount of 0.1 to 15 wt.-% of the compound(s) of the invention, relative to the weight of the composition. Typical compositions are known to the experts.

Laundry detergents typically comprise other surfactants of the anionic, nonionic, amphoteric or cationic type; builders such as phosphates, aminocarboxylates and zeolites; organic co-builders such as polycarboxylates; bleaching agents and their activators; foam controlling agents; enzymes; anti-greying agents; optical brighteners; and stabilizers.

Liquid laundry detergents generally comprise the same components as granular laundry detergents, but generally contain less of the builders. Moreover, liquid detergent formulations often comprise hydrotropic substances. All purpose cleaning agents may comprise other surfactants, builders, foam suppressing agents, hydrotropes and solubilizer alcohols.

Builders may be comprised in amounts of up to 90% by weight, preferably about 5 to 35% by weight, to intensify the cleaning action. Examples of common inorganic builders are phosphates, polyphosphates, alkali metal carbonates, silicates and sulfates. Examples of organic builders are polycarboxylates, aminocarboxylates such as ethylenediaminetetraacetates, nitrilotriacetates, hydroxycarboxylates, citrates, succinates and substituted and unsubstituted alkanedi- and polycarboxylic acids.

Another type of builder, useful in granular laundry and built liquid laundry agents, includes various substantially water-insoluble materials which are capable of reducing the water hardness, e.g., by ion exchange processes. In particular, complex sodium aluminosilicates, known as type A zeolites, are useful for this purpose.

The laundry detergents may also contain bleaching agents, e.g., percompounds such as perborates, percarbonates, persulfates and organic peroxy acids. Formulations containing percompounds may also contain stabilizing agents, such as magnesium silicate, sodium ethylenediaminetetraacetate or sodium salts of phosphonic acids. In addition, bleach activators can be used to increase the efficiency of the inorganic persalts at lower washing temperatures. Particularly useful for this purpose are substituted carboxylic acid amides, e.g., tetraacetylethylenediamine, substituted carboxylic acids, e.g., isononyloxybenzenesulfonate and sodium cyanamide.

Examples of suitable hydrotropic substances are alkali metal salts of benzene, toluene and xylene sulfonic acids; alkali metal salts of formic acid, citric and succinic acid, urea, mono-, di-, and triethanolamine. Examples of solubilizer alcohols are ethanol, isopropanol, mono- or polyethylene glycols, monopropylene glycol and ether alcohols.

Examples of foam controlling agents are high molecular weight fatty acid soaps, paraffinic hydrocarbons, and silicon containing defoamers. In particular, hydrophobic silica particles having silicon adsorbed thereon are efficient foam control agents in these laundry detergent formulations.

Examples of known enzymes which are effective in laundry detergent agents are, among others, proteases, amylases, cellulases, mannanases, and lipases. Preference is given to enzymes which have their optimum performance at the design conditions of the detergent.

A large number of fluorescent whiteners are described in the literature. For laundry detergent formulations, the derivatives of diaminostilbene disulfonates and substituted distyrylbiphenyl are particularly suitable.

As anti-greying agents, water-soluble colloids of an organic nature are preferably used. Examples are water-soluble polyanionic polymers such as polymers and copolymers of acrylic and maleic acid, cellulose derivatives such as carboxymethyl cellulose, methyl- and hydroxyethylcellulose.

In addition to one or more of the aforementioned other surfactants and other detergent composition components, laundry detergent compositions typically comprise one or more inert components. For instance, the balance of liquid detergent composition is typically an inert solvent or diluent, most commonly water.

Manual dishwashing products may comprise, besides the compounds obtained in the process of the invention, other surfactants of the anionic, nonionic, amphoteric or cationic type; solvents; diamines; carboxylic acids or salts thereof; polymeric suds stabilizers; enzymes; builders; perfumes; and/or chelating agents.

Suitable solvents include diols, polymeric glycols, and mixtures thereof. Preferred diols include propylene glycol, 1,2 hexanediol, 2-ethyl-1,3-hexanediol and 2,2,4-trimethyl-1,3-pentanediol.

Polymeric glycols, which comprise ethylene oxide (EO) and propylene oxide (PO) groups, may also be included in the present invention. These materials are formed by adding blocks of ethylene oxide moieties to the ends of polypropylene glycol chains. A preferred polymeric glycol is a polypropylene glycol having an average molecular weight in the range of 1,000 to 5,000 g/mol. When polymeric glycols are present, it may be beneficial to include either a diol and/or an alkali metal inorganic salt, such as sodium chloride, so as to obtain satisfactory physical stability. Suitable amounts of diols to provide physical stability are in the amounts in the ranges found above.

Further suitable solvents include glycols or alkoxylated glycols, ethers and diethers having from 4 to 14 carbon atoms, preferably from 6 to 12 carbon atoms, aromatic alcohols, alkoxylated aromatic alcohols, aliphatic branched alcohols, alkoxylated aliphatic branched alcohols, linear C₁-C₅ alcohols, alkoxylated linear C₁-C₅ alcohols, C₈-C₁₄ alkyl and cycloalkyl hydrocarbons and halo hydrocarbons, C₆-C₁₆ glycol ethers, and mixtures thereof.

Suitable alkoxylated glycols are methoxy octadecanol and/or ethoxyethoxyethanol. Suitable aromatic alcohols include benzyl alcohol. Suitable aliphatic branched alcohols include 2-ethylbutanol and/or 2-methylbutanol. Suitable alkoxylated aliphatic branched alcohols include 1-methylpropoxyethanol and/or 2-methylbutoxyethanol. Suitable linear C₁-C₅ alcohols include methanol, ethanol, and/or propanol.

Manual dishwashing compositions typically comprise 0.01 to 20 wt.-% of solvent, based on the total weight of the composition. The solvents may be used in conjunction with an aqueous liquid carrier, such as water, or they may be used without any aqueous liquid carrier being present.

Manual dishwashing compositions may further comprise one or more diamines.

The composition preferably comprises 0.1 to 15 wt.-% of at least one diamine, based on the total weight of the composition.

Suitable diamines include organic diamines in which pK1 and pK2 are in the range of 8.0 to 11.5, such as 1,3-bis(methylamine)-cyclohexane (pKa=10 to 10.5), 1,3-propane diamine (pK1=10.5; pK2=8.8), 1,6-hexane diamine (pK1=11; pK2=10), 1,3-pentane diamine (pK1=10.5; pK2=8.9), 2-methyl-1,5-pentane diamine (Dytek A) (pK1=11.2; pK2=10.0). pKa values referenced herein may be obtained from literature, such as from “Critical Stability Constants: Volume 2, Amines” by Smith and Martel, Plenum Press, NY and London, 1975. The pKa of the diamines is specified in an all-aqueous solution at 25° C. and for an ionic strength between 0.1 to 0.5 M.

Other preferred materials are primary diamines having two primary amino groups with alkylene spacers ranging from C₄ to C₈.

The compositions according to the present invention may comprise a linear or cyclic carboxylic acid or salt thereof. Where the acid or salt thereof is present and is linear, it preferably comprises from 1 to 6 carbon atoms whereas where the acid is cyclic, it preferably comprises greater than 3 carbon atoms. The linear or cyclic carbon-containing chain of the carboxylic acid or salt thereof may be substituted with a substituent group selected from the group consisting of hydroxyl, ester, ether, aliphatic groups having from 1 to 6, more preferably 1 to 4 carbon atoms and mixtures thereof.

Preferred carboxylic acids are those selected from the group consisting of salicylic acid, maleic acid, acetyl salicylic acid, 3-methyl salicylic acid, 4-hydroxy isophthalic acid, dihydroxyfumaric acid, 1,2,4-benzene tricarboxylic acid, pentanoic acid and salts thereof and mixtures thereof. Where the carboxylic acid exists in the salt form, the cation of the salt is preferably selected from alkali metal, alkaline earth metal, monoethanolamine, diethanolamine or triethanolamine and mixtures thereof.

The carboxylic acid or salt thereof is preferably present in an amount from 0.1% to 5 wt.-%, based on the total weight of the composition.

The composition may comprise a polymeric suds stabilizer. These polymeric suds stabilizers provide extended suds volume and suds duration without sacrificing the grease cutting ability of the liquid detergent compositions. Suitable polymeric suds stabilizers include homopolymers of (N, N-di(C₁-C₈ alkyl)amino)(C₁-C₈)alkyl acrylate esters; and copolymers thereof.

The molecular weight of the polymeric suds stabilizers is preferably in the range of 1,000 to 2,000,000 g/mol, most preferably from 20,000 to 500,000 g/mol. Polymeric suds stabilizer may be present in the form of a salt, for example the citrate, sulfate, or nitrate salt of (N,N-dimethylamino)alkyl acrylate ester. One preferred polymeric suds stabilizer is (N,N-dimethylamino)alkyl acrylate ester. Polymeric suds stabilizers are preferably present in an amount of 0.01% to 15 wt.-%, based on the total weight of the composition.

Suitable builders include aluminosilicate materials, silicates, polycarboxylates and fatty acids, materials such as ethylene-diamine tetraacetate, metal ion sequestrants such as aminopolyphosphonates, particularly ethylenediamine tetramethylene phosphonic acid and diethylene triamine pentamethylene-phosphonic acid. Though less preferred for obvious environmental reasons, phosphate builders can also be used.

Suitable polycarboxylate builders include citric acid, preferably in the form of a water-soluble salt, and derivatives of succinic acid. Specific examples include lauryl succinate, myristyl succinate, palmityl succinate 2-dodecenylsuccinate, 2-tetradecenyl succinate.

Succinate builders are preferably used in the form of their water-soluble salts, including sodium, potassium, ammonium and alkanolammonium salts. Other suitable polycarboxylates are oxodisuccinates and mixtures of tartrate monosuccinic and tartrate disuccinic acid, as described in U.S. Pat. No. 4,663,071.

Suitable fatty acid builders include saturated and unsaturated C₁₀₋₁₈ fatty acids, as well as the corresponding soaps. Preferred saturated species have from 12 to 16 carbon atoms in the alkyl chain. The preferred unsaturated fatty acid is oleic acid. Other preferred builder system for liquid compositions is based on dodecenyl succinic acid and citric acid.

Builders are preferably present in amounts of 0.5% to 50 wt.-%, more preferably 5 to 25 wt.-%, based on the total weight of the composition.

Suitable enzymes include enzymes selected from cellulases, hemicellulases, peroxidases, proteases, gluco-amylases, amylases, lipases, cutinases, pectinases, xylanases, reductases, oxidases, phenoloxidases, lipoxygenases, ligninases, pullulanases, tannases, pentosanases, malanases, β-glucanases, arabinosidases or mixtures thereof. Enzymes may be present in amounts of 0.0001% to 5 wt.-% of active enzyme, based on the total weight of the composition.

Preferred proteolytic enzymes, then, are selected from the group consisting of Alcalase® (Novo Industri A/S), BPN′, Protease A and Protease B (Genencor), and mixtures thereof. Protease B is most preferred. Preferred amylase enzymes include TERMAMYL®, DURAMYL® and the amylase enzymes those described in WO 9418314 to Genencor International and WO 9402597 to Novo.

Suitable chelating agents include iron and/or manganese chelating agents. Such chelating agents can be selected from the group consisting of amino carboxylates, amino phosphonates, polyfunctionally-substituted aromatic chelating agents and mixtures thereof. Suitable amino carboxylates include ethylenediaminetetraacetates, N-hydroxyethylethylenediaminetriacetates, nitrilo-tri-acetates, ethylenediamine tetrapro-prionates, triethylenetetraaminehexaacetates, diethylenetriaminepentaacetates, and ethanoldiglycines, alkali metal, ammonium, and substituted ammonium salts thereof, and mixtures thereof. Suitable amino phosphonates include ethylenediaminetetrakis (methylenephosphonates). Suitable polyfunctionally-substituted aromatic chelating agents include dihydroxydisulfobenzenes such as 1,2-dihydroxy-3,5-disulfobenzene. Chelating agents may be present in amounts of 0.00015% to 15 wt.-%, based on the weight of the composition.

The invention is described in further detail by the subsequent examples.

### EXAMPLES

### General Information

Reductive aminations of an aldehyde with a secondary amine were performed at a temperature of 25 to 50° C. and at a hydrogen pressure of 5 to 10 bar in a 300 mL autoclave in accordance with Table 1 below.

Analysis was performed via gas chromatography (after silylation) or via gas chromatography-mass spectrometry (GC-MS), and structures were confirmed by nuclear magnetic resonance (NMR) spectroscopy.

### Example 1

Palladium on carbon black (10 wt.-%, 1.9 g) was added to a solution of pyrrolidine (99%, 10 g) and dodecanal (95%, 29 g) in tetrahydrofuran (100 mL) in an autoclave. The reaction vessel was closed and subsequently purged with nitrogen gas (thrice at 5 bar). At an initial hydrogen gas pressure of 5 bar, stirring (700 rpm) was applied. The reaction mixture was warmed to 25° C. and the hydrogen pressure was maintained at 5 bar.

The reaction mixture was stirred under these conditions for 12 h, then cooled to room temperature and purged with nitrogen gas (thrice at 5 bar). Afterwards, the catalyst was filtered off and the solvent was removed by evaporation, yielding the crude material as a liquid (purity: 71%, 35 g, yield: 75%).

### Example 2

Example 2 was performed analogously to Example 1, as indicated in Table 1.

### Example 3

Palladium on carbon black (10 wt.-%, 1 g) was added to a solution of morpholine (99%, 10 g) in ethanol (90 mL) in an autoclave. The reaction vessel was closed and subsequently purged with nitrogen gas at (thrice at 5 bar). At an initial hydrogen gas pressure of 5 bar, stirring (700 rpm) was applied. The reaction mixture was warmed to 40° C. Subsequently, dodecanal (95%, 25 g) as a solution in ethanol (20 mL) was metered continuously to the reaction mixture over the course of 5 h. The temperature (40° C.) and the hydrogen pressure (5 bar) were maintained.

After complete addition of the aldehyde solution, the reaction mixture was stirred at a temperature of 40° C. and a hydrogen pressure of 5 bar for 1 h, then cooled to room temperature and purged with nitrogen gas (thrice at 5 bar). Afterwards, the catalyst was filtered off and the solvent was removed by evaporation, yielding the crude material as a colourless liquid (purity: 91%, 29 g, yield: 91%).

### Example 4

Palladium on carbon black (10 wt.-%, 1.5 g) was added to a solution of pyrrolidine (99%, 15 g) in a mixture of ethanol (50 mL) and water (40 mL) in an autoclave. The reaction vessel was closed and subsequently purged with nitrogen gas at (thrice at 5 bar). At an initial hydrogen gas pressure of 5 bar, stirring (700 rpm) was applied. The reaction mixture was warmed to 40° C. Subsequently, dodecanal (95%, 45 g) as a solution in ethanol (20 mL) was metered continuously to the reaction mixture over the course of 5 h. The temperature (40° C.) and the hydrogen pressure (5 bar) were maintained.

After complete addition of the aldehyde solution, the reaction mixture was stirred at a temperature of 40° C. and a hydrogen pressure of 5 bar for 1 h, then cooled to room temperature and purged with nitrogen gas (thrice at 5 bar). Afterwards, the catalyst was filtered off and the solvent was removed by evaporation, yielding the crude material as a yellow liquid (purity: 89%, 46 g, yield: 80%). Distillation (5 mbar, 140-150° C.) yielded the product as a colorless liquid (purity: 99%, 30 g, yield: 59%).

### Examples 5 to 8

Examples 5 to 8 were performed analogously to Example 4, as indicated in Table 1.

### Example 9

Palladium on carbon black (10 wt.-%, 1.5 g) was added to a solution of N-methyl ethanolamine (100%, 15 g) and dodecanal (95%, 43 g) in a mixture of ethanol (70 mL) 5 and water (40 mL) in an autoclave. The reaction vessel was closed and subsequently purged with nitrogen gas (thrice at 5 bar). At an initial hydrogen gas pressure of 20 bar, stirring (700 rpm) was applied. The reaction mixture was warmed to 50° C. and the hydrogen pressure was maintained at 20 bar.

The reaction mixture was stirred under these conditions for 6 h, then cooled to room temperature and purged with nitrogen gas (thrice at 5 bar). Afterwards, the catalyst was filtered off and the solvent was removed by evaporation, yielding the crude material as a liquid (purity: 93%, yield: 88%).

TABLE 1

| | | Solvent | | | | | | | | | |
|---|---|---|---|---|---|---|---|---|---|---|---|
| | | Dilution | δ ² | | Secondary | | Time | H₂ | T | Yield ³ | Purity ³ |
| # | Solvent | Ratio ¹ | [MPa^{1/2}] | Catalyst | Amine | Aldehyde | [h] | [bara] | [° C.] | [%] | [%] |
| 1 * | THF | 2.6 | 18.5 | Pd/C ⁴ | pyrrolidine | dodecanal | 12 | 5 | 25 | 75 | 71 |
| | (100 mL) | | | (1.9 g) | (10 g) | (29 g) | | | | | |
| 2 * | THF | 3.0 | 18.5 | Pd/C ⁴ | piperidine | dodecanal | 12 | 5 | 30 | 75 | 77 |
| | (100 mL) | | | (2.3 g) | (10 g) | (23 g) | | | | | |
| 3 | EtOH | 3.2 | 26.2 | Pd/C ⁴ | morpholine | dodecanal | 5 + 1 ⁵ | 5 | 40 | 91 | 91 |
| | (110 mL) | | | (1.0 g) | (10 g) | (25 g) | | | | | |
| 4 | EtOH/water | 1.8 | 34.1 | Pd/C ⁴ | pyrrolidine | dodecanal | 5 + 1 ⁵ | 5 | 40 | 80 | 89 |
| | 1.75:1 | | | (1.5 g) | (15 g) | (45 g) | | | | | |
| | (110 mL) | | | | | | | | | | |
| 5 | EtOH/water | 1.9 | 34.1 | Pd/C ⁴ | N-methyl | dodecanal | 5 + 1 ⁵ | 20 | 50 | 93 | 91 |
| | 1.75:1 | | | (2.0 g) | ethanolamine | (43 g) | | | | | |
| | (110 mL) | | | | (15 g) | | | | | | |
| 6 | EtOH/water | 1.7 | 34.1 | Pd/C ⁴ | N-methyl | citral ⁶ | 5 + 1 ⁵ | 20 | 50 | 94 | 96 |
| | 1.75:1 | | | (2.0 g) | ethanolamine | (45 g) | | | | | |
| | (110 mL) | | | | (20 g) | | | | | | |
| 7 | EtOH/water | 1.8 | 34.1 | Pt/C ⁷ | pyrrolidine | dodecanal | 5 + 1 ⁵ | 5 | 40 | 99 | 85 |
| | 1.75:1 | | | (8.0 g) | (15 g) | (45 g) | | | | | |
| | (110 mL) | | | | | | | | | | |
| 8 * | EtOH/water | 1.9 | 34.1 | Pd/C ⁴ | N-methyl | dodecanal | 5 + 1 ⁵ | 50 | 80 | 92 | 79 |
| | 1.75:1 | | | (2.0 g) | ethanolamine | (43 g) | | | | | |
| | (110 mL) | | | | (15 g) | | | | | | |
| 9 * | EtOH/water | 1.9 | 34.1 | Pd/C ⁴ | N-methyl | dodecanal | 6 | 20 | 50 | 93 | 88 |
| | 1.75:1 | | | (2.0 g) | ethanolamine | (43 g) | | | | | |
| | (110 mL) | | | | (15 g) | | | | | | |

¹ volume contractions were taken into account for mixtures of two solvents
² Hildebrandt solubility parameter
³ yield and purity refer to the crude product
⁴ 10 wt-% palladium on carbon black (no water content)
⁵ time for continuous dosing of the aldehyde, plus additional stirring time
⁶ 3,7-dimethylocta-2,6-dienal (mixture of isomers)
⁷ 5 wt-% platinum on carbon black (50 wt-% water content)
* comparative example

### Example 10-Salt Form of Example 1

Crude N-dodecyl pyrrolidine as obtained in example 1 (71%, 10 g) was dissolved in tetrahydrofuran (50 mL). Concentrated aqueous hydrochloric acid (37%) was added to the solution until a pH value of 1 was reached. The reaction mixture was stirred at room temperature for 12 h. Afterwards, the solvent was removed by evaporation, and the crude material was recrystallized from heptane/toluene (19:1) at 75° C. (purity: 82%, 10 g, yield: 84%).

1. A process for producing a tertiary amine surfactant of general formula (I) 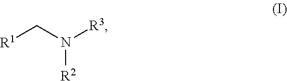
or a salt thereof, wherein
R¹ is selected from the group consisting of C₅-C₁₇-alkyl and C₅-C₁₇-alkenyl;
R² and R³ are independently selected from the group consisting of C₁-C₈-alkyl and C₃-C₈-alkenyl, wherein C₁-C₈-alkyl and C₃-C₈-alkenyl are each optionally substituted with one or more substituents selected from the group consisting of a hydroxy group, a C₁-C₈-alkoxy group, an amino group, a C₁-C₈-alkylamino group, a thio group and a C₁-C₈-alkylthio group;
or wherein R² and R³, together with the nitrogen atom to which they are bound, form a 5- or 6-membered heterocycle, optionally substituted with one or more substituents selected from the group consisting of a hydroxy group, a C₁-C₈-alkoxy group, an amino group, a C₁-C₈-alkylamino group, a thio group and a C₁-C₈-alkylthio group;
the process comprising reductive amination of an aldehyde of general formula (II) 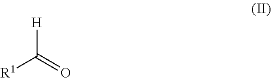
wherein R¹ is defined as above;
with a secondary amine of general formula (III) 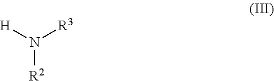
wherein R² and R³ are defined as above;
in the presence of molecular hydrogen and a heterogeneous catalyst comprising a group 10 element of the periodic table of the elements;
wherein the reductive amination is conducted in feed operation, wherein the secondary amine of general formula (III) is provided, and the aldehyde of general formula (II) is metered thereto; and
wherein the reductive amination is performed at a pressure of molecular hydrogen in the range of at least 1 bara to less than 40 bara; and at a temperature in the range of 25 to 70° C.

2. The process according to claim 1, wherein R¹ is C₇-C₁₇-alkyl; or wherein R¹ is C₉-C₁₇-alkenyl.

3. The process according to claim 1, wherein R² is optionally substituted C₁-C₃-alkyl; and R³ is optionally substituted C₁-C₃-alkyl.

4. The process according to claim 1, wherein R² and R³, together with the nitrogen atom to which they are bound, form a pyrrolidinyl group, a piperidinyl group, a morpholinyl group, or a 8-oxa-3-azabicyclo[3.2.1]octanyl group, each of which is optionally substituted.

5. The process according to claim 1, wherein the group 10 element is selected from the group consisting of palladium, platinum and nickel.

6. The process according to claim 5, wherein the heterogeneous catalyst is palladium on activated carbon.

7. The process according to claim 1, wherein the process is conducted in the presence of a solvent.

8. The process according to claim 7, wherein the solvent comprises a protic solvent and/or an aprotic solvent comprising an ether moiety.

9. The process according to claim 8, wherein the solvent is selected from the group consisting of an aliphatic alcohol and a mixture of an aliphatic alcohol and water.

10. The process according to claim 7, wherein the solvent dilution ratio is less than 5.0 L per kg, the solvent dilution ratio being the total volume of solvent to the total weight of the aldehyde of general formula (II) and the secondary amine of general formula (III).

11. The process according to claim 1, comprising neutralizing the tertiary amine surfactant of general formula (I) with an acid.

12. The process according to claim 1, wherein the aldehyde of general formula (II) and/or the secondary amine of general formula (III) are bio-based compounds.

13. A surfactant composition, comprising a surfactant of general formula (I) or a salt thereof as defined according to claim 1 in an amount of at least 90 wt.-%, relative to the weight of the surfactant composition, and a compound of general formula (IV) 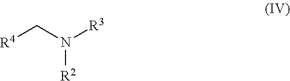
or a salt thereof in an amount of 0.01 to 5.0 wt.-%, relative to the weight of the surfactant composition,
wherein R² and R³ are defined as above; and R⁴ is selected from the group consisting of C₁₀-C₃₄-alkyl and C₁₀-C₃₄-alkenyl, with the proviso that the number of carbon atoms in R⁴ differs from the number of carbon atoms in R¹ of the surfactant of general formula (I).

14. A compound selected from the group consisting of
2-[(3,7-dimethyloctyl)(methyl)amino]ethanol,
1-(3,7-dimethyloctyl)pyrrolidine,
3-dodecyl-8-oxa-3-azabicyclo[3.2.1]octane,
3-nonyl-8-oxa-3-azabicyclo[3.2.1]octane, and
3-(3,7-dimethyloctyl)-8-oxa-3-azabicyclo[3.2.1]octane,
or a salt thereof.

15. A composition comprising at least one compound according to claim 14.

16. A method of using the compound according to claim 15, the method comprising using the compound as a surfactant.

17. The process according to claim 1, wherein R¹ is a C₈-, C₉- or C₁₁-alkyl.

18. The process according to claim 1, wherein R¹ is a C₉-alkenyl.

19. The process according to claim 1, wherein R² is C₁-C₃-alkyl substituted with a hydroxy group.

20. The process according to claim 1, wherein R³ is methyl.
